(19) United States
(12) Patent Application Publication
(10) Pub. No.: US 2012/0245351 A1
(43) Pub. Date: Sep. 27, 2012

(54) PROCESS FOR THE PREPARATION OF LAPATINIB AND ITS PHARMACEUTICALLY ACCEPTABLE SALTS

(75) Inventors: Ramanadham Jyothi Prasad, Hyderabad (IN); Bhujanga rao Adibhatla Kali Satya, Hyderabad (IN); Nannapaneni Venkaiah Chowdary, Hyderabad (IN)

(73) Assignee: NATCO PHARMA LIMITED, Hyderabad (IN)

(21) Appl. No.: 13/498,389

(22) PCT Filed: Sep. 29, 2009
(86) PCT No.: PCT/IN09/00533

§ 371 Date: Jun. 11, 2012

(51) Int. Cl. C07D 239/94

(52) U.S. Cl. 544/293

(57) ABSTRACT

The present invention relates to an improved and new process for the preparation of high purity crystalline base of Lapatinib of formula (1) having chemical name N-{3-chloro-4-[(3-fluorobenzyloxy]phenyl}-6-[5-({[2-(methanesulfonyl)ethyl]amino}methyl]-2-furyl]-4-quip-azolinamine and its pharmaceutically acceptable salts.

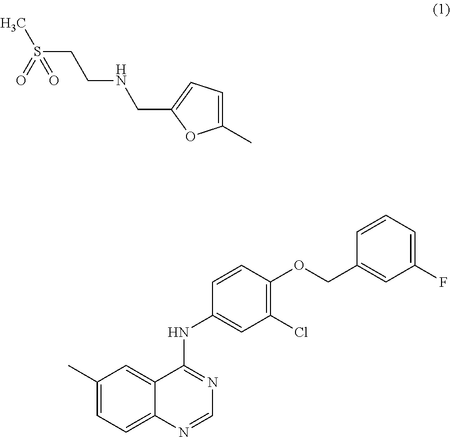

### FIELD OF INVENTION

The present invention relates to an improved and new process for the preparation of high purity crystalline base of Lapatinib of formula-(1) having chemical name N-{3-Chloro-4-[(3-fluorobenzyloxy]phenyl}-6-[5-({[2-(methanesulfonyl)ethyl]amino}methyl]-2-furyl]-4-quin-azolinamine and its pharmaceutically acceptable salts.

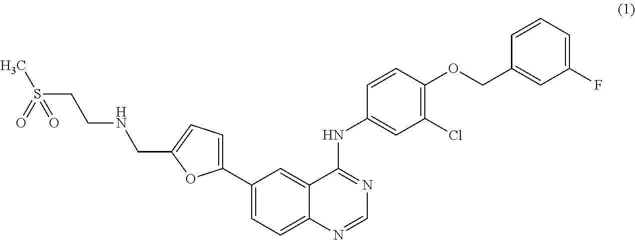

Lapatinib is a molecule that inhibits the activity of both Erb B₁ and Erb B₂ and has shown clinical activity in breast cancer. As a reversible and dual—acting inhibitor the drug will be able to overcome problems of resistance encountered with single inhibitors.

### BACKGROUND OF INVENTION

Lapatinib of formula-(1), is reported for the first time by M. C. Carter et. al in PCT International Publication No.: WO 99/35146 (1999 to Glaxo). Its equivalent US patent is U.S. Pat. No. 6,727,256 (2004 to SmithKline Beecham). Study on mechanism of action of Lapatinib is described in Oncogene, 21, 6255 (2002). In vitro anti-tumor activity in combination with anti-ErbB₂ antibodies is described in Oncogene, 24, 6213 (2005), biological effects on tumor growth is described in J. Clin. Oncol, 23, 2502 (2005) and pharmacokinetics and clinical activity in metastatic carcinomas is described in J. clin. oncol, 23, 5305 (2005).

The process for the preparation of Lapatinib of formula-(1), disclosed in WO99/35146, is given in the Scheme-A. Accordingly, 4-chloro-6-iodo-quinazoline of formula-(2), is reacted with 3-chloro-4-(3¹-fluoro-benzyloxy)-aniline yielding N-[3-chloro-4-{(3¹-fluoro-benzyloxy)phenyl}]-6-iodo-quinazoline of formula-(3). The compound of the formula-(3) reacts with (1,3-dioxolan-2-yl)-2-(tributylstannyl)furan to get the compound of formula-(4a) which on reaction with HCl, removes the protecting group and liberates 5-(4-{3-chloro-4-(3-fluoro-benzyloxy)anilino}-6-quinazolinylguran-2-carbaldehyde of formula-(4). The compound of the formula-(4) on reaction with 2-methanesulfonylethylamine, followed by reduction using sodium (triacetoxy)borohydride as the reducing agent gives the required compound Lapatinib of formula-(1) as an organic residue, which is purified by column chromatography. If desired the isolated material is then converted into the hydrochloride salt. 1(a)

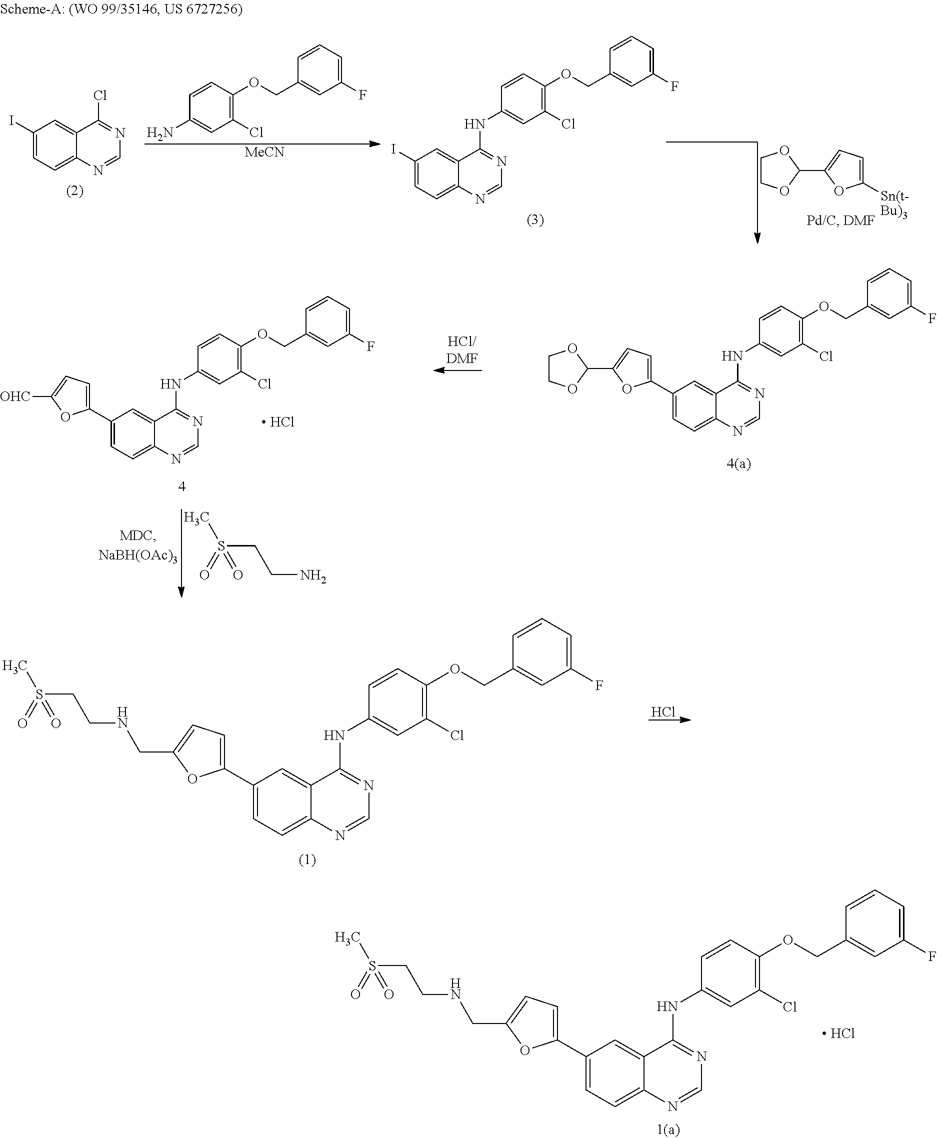

In the subsequent PCT-international publication No. WO 02/02552 (glaxo) and its equivalent U.S. Pat. No. 7,157,466; the preparation of ditosylate salts of Lapatinib of formula-1(b) is disclosed as shown in Scheme-B.

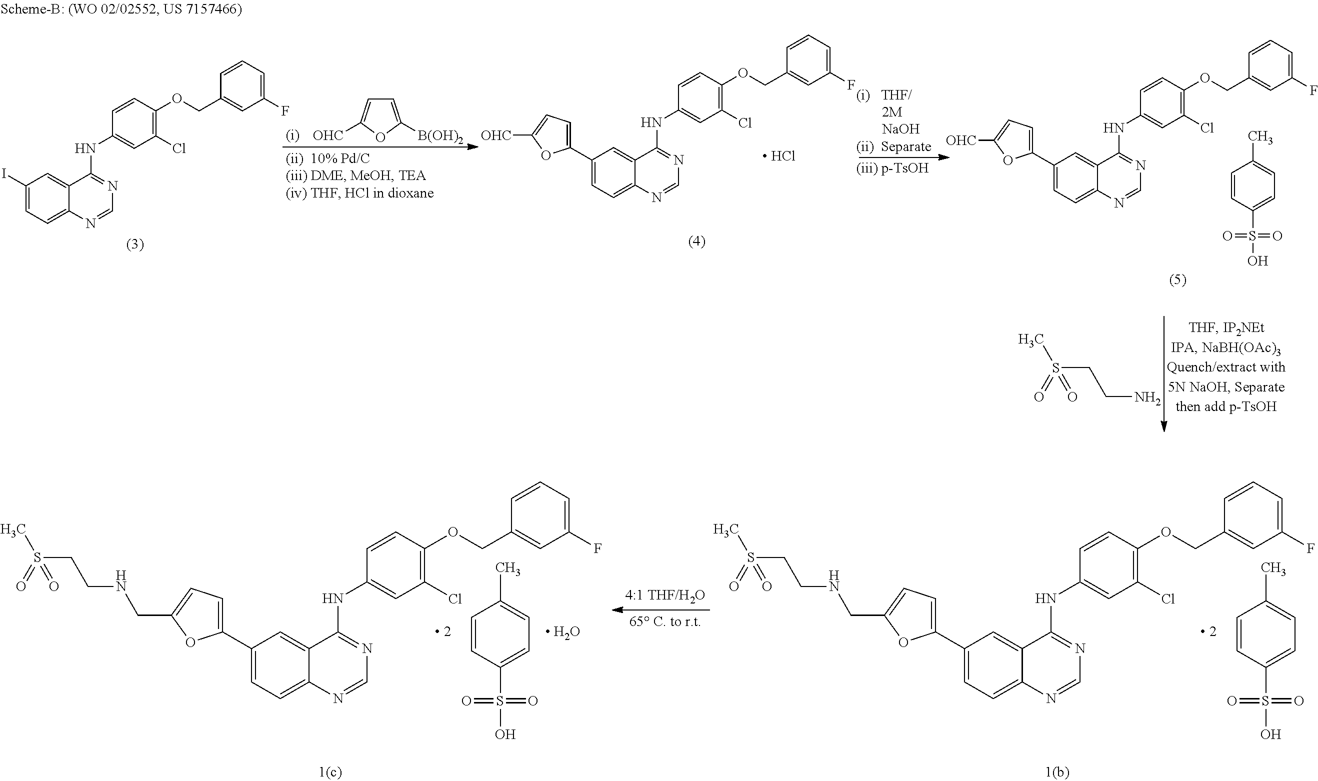

In both of these patents, the process involves multiple steps to get the required product. The process is lengthy and cumbersome and also involves usage of corrosive chemicals like POCl₃/SOCl₂ etc.

### SUMMARY OF INVENTION

Keeping in view of the difficulties in the above mentioned prior art processes for the preparation of Lapatinib on a commercial scale, we aimed to develop a simple and economically viable and commercially applicable process for the preparation of Lapatinib, of formula-(1).

Accordingly, the main objective of the present invention is to provide an improved process for the preparation of Lapatinib of formula-(1), which is simple, economical and commercially applicable.

According to another objective of the present invention is to provide an improved process for the preparation of Lapatinib of formula-(1), which involves readily and cheaply available raw materials.

During our elaborate research in developing a process for the preparation of Lapatinib of formula-(1) on a commercially viable scale, we observed that commercially and readily available 2-aminobenzonitrile of formula-(6) could be a suitable starting material, when compared to 4-chloro-6-iodo-quinazoline of formula-(2) used in the prior art.

The preparation of key intermediate 2-amino-5-iodobenzonitrile of formula-(7) starting from 2-amino benzonitrile of formula-(6) is reported by Harris, N. V; Smith, C; et al in Eur. J. Med. Chem 1992, 27, 7-18. We adopted the same procedure with modifications at recrystallization step for the preparation of 2-amino-5-iodobenzonitrile of formula-(7).

Similarly the preparation of the compound N′-(2-cyano-4-iodo-phenyl)-N,N-dimethylforamidine of formula-(8) starting from 2-amino-5-iodobenzonitrile is reported in PCT international publication number WO 2006/079833 (Arrow therapeutics limited, GB) as a brown oil. We modified this process to get the required product as a pale yellow coloured amorphous powder having a melting range of 53-55° C. and purity of 99.8% by HPLC.

Accordingly, the present invention provides an improved process for the preparation of Lapatinib of formula-(1).

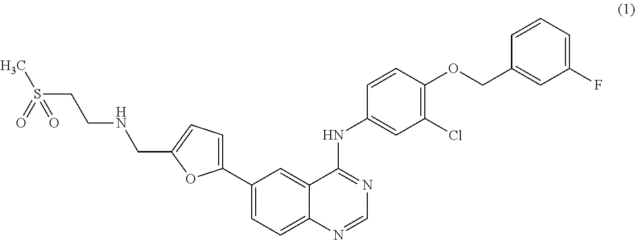

and its pharmaceutically acceptable salts, which comprises:—
(i) Reacting 2-aminobenzonitrile of formula-(6)

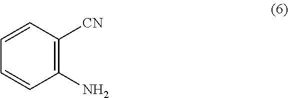

with iodinemonochloride or iodine crystals in acetic acid medium at elevated temperature to get 2-amino-5-iodobenzonitrile of the formula-(7), which is purified by recrystallization from an organic solvent or a mixture of solvents.

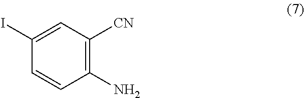

(ii) Reacting 2-amino-5-iodobenzonitrile of formula-(7) with N,N-dimethylformamide dimethyl acetal in an organic solvent and at an elevated temperature yielding the compound N′-(2-cyano-4-iodo-phenyl)-N,N-dimethyl formamidine of the formula-(8)

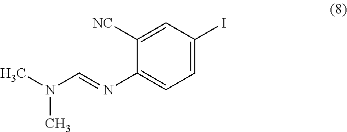

(iii) Reacting 3-chloro-4-(3-fluorobenzyloxy)aniline of formula-(8a)

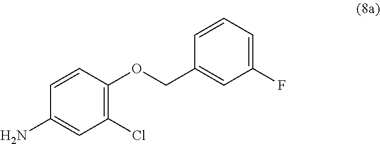

with the compound of the formula-(8) in presence of an acid catalyst and at an elevated temperature to get a compound of the formula-(3).

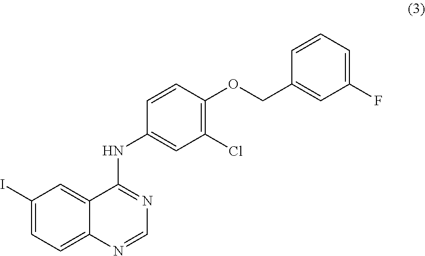

(iv) Reacting the compound of the formula-(3) with 5-formyl-2-furyl boronic acid by Palladium (O) mediated biaryl coupling (Suzuki cross coupling) in an ethereal solvent at an elevated temperature to get the desired compound of formula-(4).

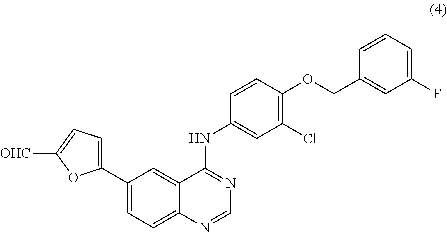

(v) Reacting the compound of the formula-(4), with 2-methanesulfonylethylamine or its salt in a suitable solvent, at an elevated temperature gives the imine compound of the formula-(9).

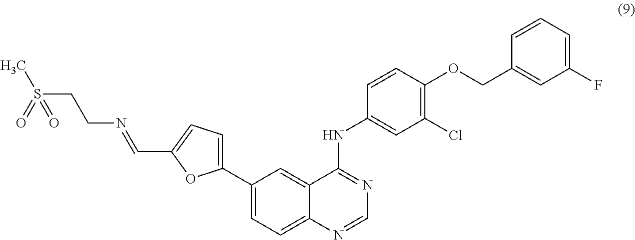

(vi) Reacting the compound of the formula-(9) with a suitable reducing agent in a suitable solvent and the resultant amine formed is extracted with a suitable solvent and subsequent evaporation of the solvent gives Lapatinib base of the formula-(1)

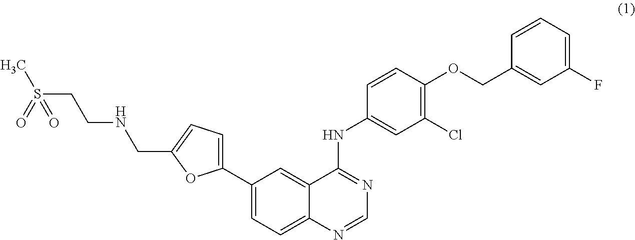

(vii) Crystallizing the crude Lapatinib base of formula-(1) from a suitable solvent to get pure Lapatinib base.
(viii) Reacting pure Lapatinib base of formula-(1) by dissolving or suspending in an organic solvent with p-toluenesulfonic acid monohydrate to get Lapatinib ditosylate (anhydrous) of formula-1(b)

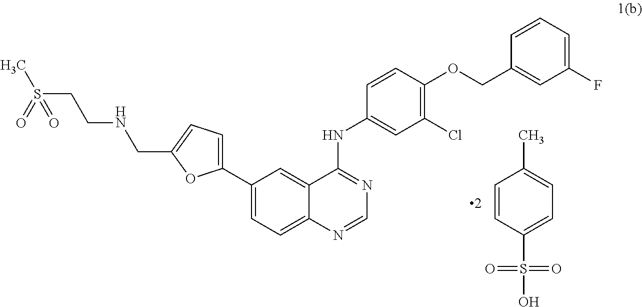

(ix) Recrystallization of Lapatinib ditosylate (anhydrous) in aqueous alcohol affords pharmaceutically acceptable grade Lapatinib ditosylate monohydrate of formula-1(c).

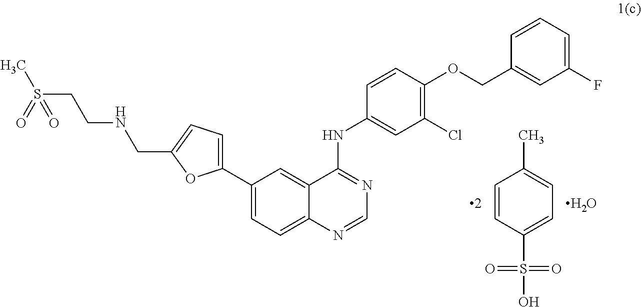

The reaction scheme of the present invention is as given in the following Scheme-(C).

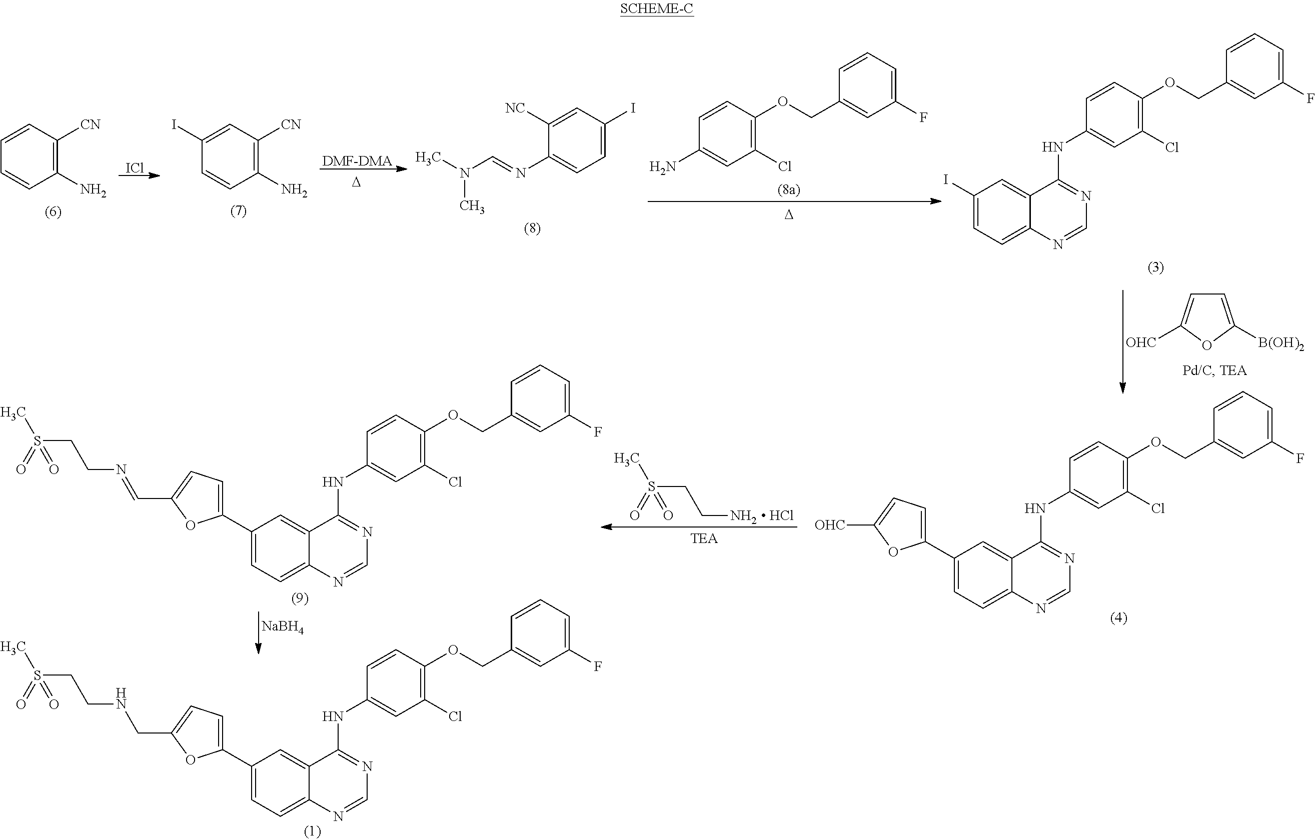

Accordingly, the basic raw material selected for the synthesis of Lapatinib of formula-(1) is commercially available 2-amino benzonitrile of formula-(6), which reacts with iodine or iodinemonochloride to get 2-amino-5-iodobenzonitrile of the formula-(7). The compound of the formula-(7) on reaction with N,N-dimethylforamamide dimethyl acetal at elevated temperature gives the compound N′-(2-cyano-4-iodo-phenyl)-N,N-dimethyl formamidine of formula-(8). This compound of the formula-(8) on reaction with 3-chloro-4-(3-fluorobenzyloxy)aniline of formula-(8a) at elevated temperature gives the compound N-[3-chloro-4-[(3-fluorobenzyloxy)phenyl]-6-iodo-quinazolinamine of formula-(3). The compound of formula-(3) on reaction with 5-formyl-2-furyl-boronic acid, in presence of triethylamine and Pd/C affords the compound 5-[4-[3-chloro-4-(3-fluorobenzyloxy)anilino]-6-quinazolinylguran-2-carbaldehyde of the formula-(4).

The compound of the formula-(4), on reaction with 2-methanesulfonylethylamine hydrochloride yields the compound N[3-chloro-4[(3-fluorobenzyloxy]phenyl]-6-[5-({[2-methanesulphonyl)-ethyl]imino}-2-furyl]-4-quinazolinamine of the formula-(9). The imine compound of formula-(9) on reduction, using sodium borohydride gives the compound of the formula-(1), which is Lapatinib base.

### BRIEF DESCRIPTION OF THE DRAWINGS

FIG. 1: Illustrates the powder X-ray diffraction pattern of Lapatinib base

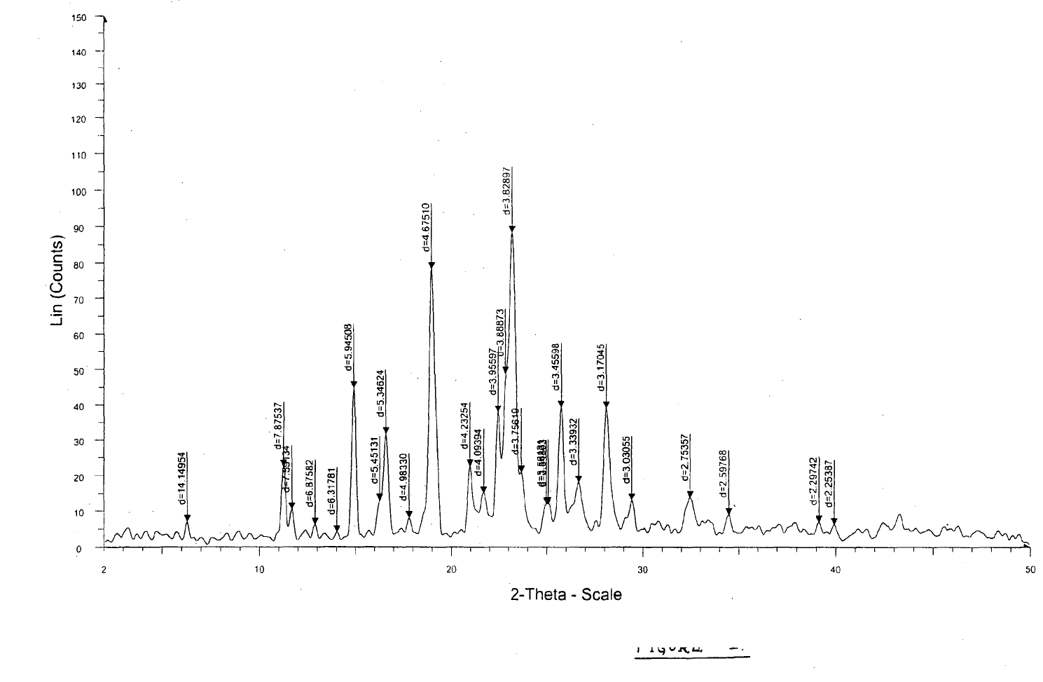

FIG. 2: Illustrates DSC thermogram of Lapatinib base

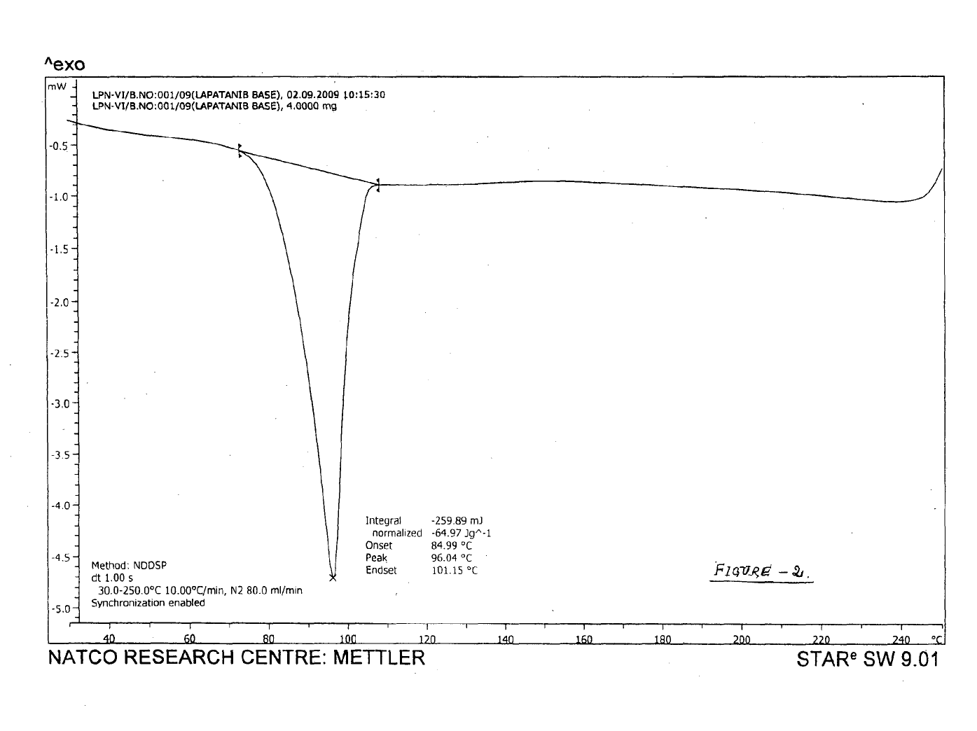

FIG. 3: Illustrates IR pattern of Lapatinib base

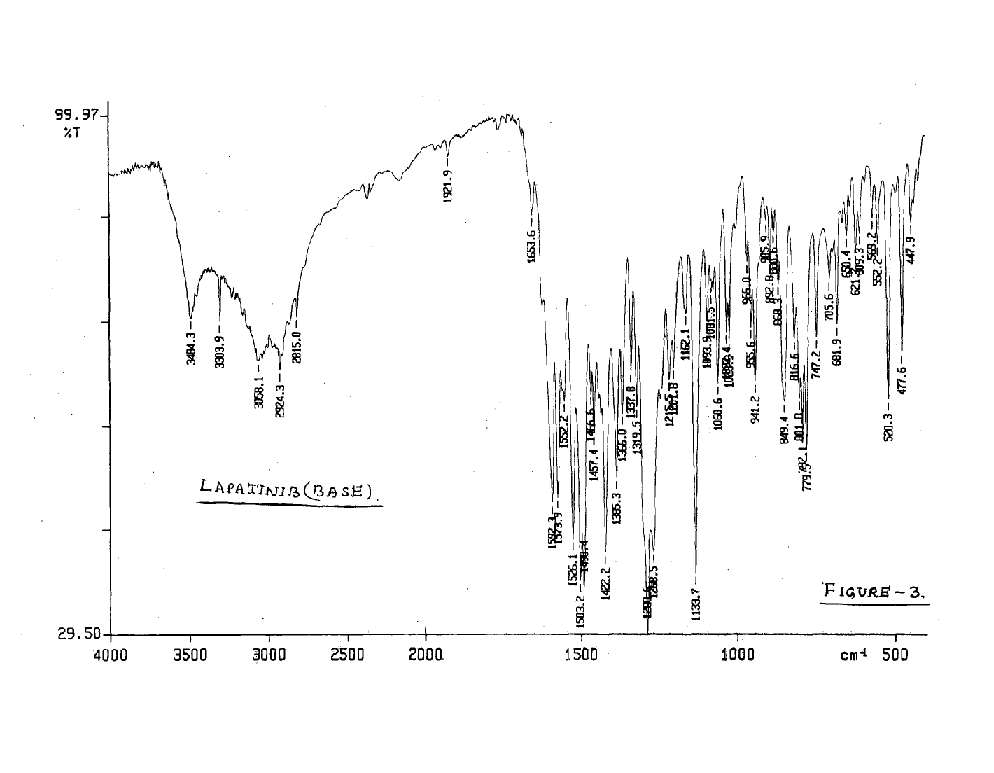

### DETAILED DESCRIPTION OF INVENTION

In a preferred embodiment of the present invention (SCHEME-C),

In the step (i), 2-aminobenzonitrile of the formula-(6) is reacted with iodinemonochloride in acetic acid medium to get the compound of the formula-(7). During the reaction, the reaction temperature is maintained at 0 to 100° C., preferably between 10 to 50° C., most preferably between 25 to 35° C. The organic solvent used for purification by recrystallization, is a mixture of toluene and hexane.

In the step (ii), the reaction of 2-amino-5-iodobenzonitrile of formula-(7), with N,N-dimethylformamide dimethylacetal may be carried out in presence of a suitable solvent or diluent, for example in an aromatic solvent such as toluene, xylene, cumene or chlorobenzene, or in a polar aprotic solvent such as acetonitrile, propionitrile, butyronitrile, ethylacetate, tetrahydrofuran, 2-methyl tetrahydrofuran, 1,4-dioxan or a dipolar aprotic solvent such as N,N-dimethylformamide, N,N-dimethyacetamide, N-methylpyrrolidin-2-one or dimethylsulfoxide. A further suitable solvent or diluent is water or a polar protic solvent such as a primary secondary or tertiary alkyl alcohol, for example, methanol, ethanol, 2-propanol, a butanol or pentanol. Mixtures of such suitable solvents or diluents may be used.

Conveniently, the reaction is carried out in an organic solvent, for example N,N-dimethylformamide, N,N-dimethyacetamide, N-methylpyrrolidin-2-one or dimethylsulfoxide preferably N,N-dimethylformamide and at a temperature in between 30 to 150° C., preferably between 70 to 80° C.

The product is obtained by quenching the reaction mass into water. The product isolation temperature is in between 0 to 40° C., preferably 0 to 5° C.

In the step (iii), the compound of formula-(8a) is coupled with compound of formula-(8) in the presence of acid catalyst which is selected from trifluoro acetic acid, formic acid or acetic acid, preferably acetic acid at a temperature range between 30-140° C., preferably 115-120° C.

The product is obtained by quenching the reaction mass into water and adjusted the pH to basic by adding aqueous ammonia or dilute caustic lye solution. The product isolation temperature is in between 0-40° C. preferably 0-5° C.

In the step (iv), in Suzuki coupling reaction, the ethereal solvent used is selected from diethyl ether, tetrahydrofuran, 1,4-dioxane, 1,2-diethoxyethane and 1,2-dimethoxy ethane, preferably 1,2-dimethoxy ethane (DME).

The catalyst used is selected from a list that includes palladium (II) acetate, palladium (II) chloride, palladium on carbon, preferably palladium on carbon.

The reaction temperature is in between 25 to 120° C. preferably between 25 to 75° C. and most preferably between 45-50° C.

In the step-(v), the aldehyde compound of the formula-(4) is reacted with 2-methanesulphonyl ethylamine or its salts with acids like HCl, HBr or H₂SO₄, preferably HCl salt. The solvent used for the reaction includes dichloroethane, dichloromethane, tetrahydrofuran, 2-methyl tetrahydrofuran, N,N-dimethyl Formamide, 1,2-dimethoxyethane and alcohols like ethanol, methanol, 2-propanol or a mixture thereof. The preferred solvents are tetrahydrofuran and methanol, most preferably methanol.

The reaction temperature is in between 0 to 125° C. preferably between 25 to 100° C. and most preferably the reflux temperature of methanol.

In the step-(vi), for the reduction of imine of formula-(9) to amine, the reducing agent used is selected from sodium triacetoxyborohydride, sodium borohydride etc, preferably sodium borohydride.

Solvent used in the reaction can be selected from tetrahydrofuran, acetonitrile, acetone, dimethylformamide, dimethylacetamide, 1,2-diethoxyethane, 1,2-dimethoxyethane or a mixture thereof, preferably a mixture of tetrahydrofuran and methanol. The reaction temperature is in between 0 to 100° C. preferably 0 to 40° C. most preferably 0 to 15° C.

The reaction mass is quenched into water and extracted with solvents like ethylacetate, methylacetate, isopropylacetate, dichloroethane, dichloromethane, chloroform, tertiary butyl methyl ether etc. preferably ethylacetate. The crude product is obtained by solvent evaporation.

In the step-(vii), the obtained crude Lapatinib base is purified by crystallization from different solvents like ethylacetate, methylacetate, isopropyl acetate, acetonitrile, methanol, ethanol, isopropanol, acetone, methylethylketone, methylenechloride, toluene, chloroform, 1,4-dioxane, dimethylfromamide, tetrahydrofuran, 2-methyltetrahydrofuran, dimethylacetamide, 1,2-dimethoxyethane, tertiarybutylmethyl ether, water or a mixture thereof, preferably ethylacetate, isopropanol and methanol.

The isolation temperature of recrystallized Lapatinib base is 0 to 35° C. preferably 25-35° C.

The purity of Lapatinib base obtained according to process of the present invention is more than 99.5% by HPLC.

The melting point range of the pure Lapatinib base obtained is 95-98° C. (peak max. by DSC)

The IR spectral values of pure Lapatinib base obtained are 3484, 3304, 3058, 2924, 2815, 1922, 1653, 1592, 1574, 1552, 1526, 1503, 1490, 1457, 1422, 1385, 1366, 1338, 1319, 1288, 1268, 1215, 1201, 1162, 1133, 1094, 1060, 1028, 955, 941, 892, 868, 849, 779, 747, 681, 650, 621, 552, 520, 477 cm⁻¹.

The principle 2θ values of powder XRD of pure Lapatinib base obtained are 11.22, 14.88, 16.56, 18.96, 20.97, 21.69, 22.45, 22.85, 23.21, 23.66, 25.75, 26.67, 28.12, 32.49

In the step-(viii), the purified Lapatinib base so obtained can be converted into ditosylate salt (anhydrous) by suspending or dissolving the Lapatinib base in an organic solvent or a mixture of organic solvents and then treating with p-toluenesulfonic acid monohydrate.

The organic solvent used for dissolving or suspending the Lapatinib base is selected from toluene, chloroform, isopropanol, ethanol, methanol, acetone, methyethylketone, acetonitrile methylacetate, ethylacetate, isopropylacetate, dimethylformamide, dimethylether, diethylether, tertiarybutylmethylether, tetrahydrofuran, 2-methyltetrahydrofuran, dimethylacetamide, 1,2-diethoxyethane, 1,2-dimethoxyethane or a mixture thereof, preferably tetrahydrofuran, methanol or most preferably methanol.

The reaction temperature during ditosylate salt formation is in between 0 to 80° C., preferably the refluxing temperature of the solvent used. The isolation temperature is in between 0 to 35° C. preferably 25-35° C.

In step-(ix), Lapatinib ditosylate monohydrate is obtained by suspending or dissolving Lapatinib ditosylate (anhydrous) in a mixture of water and organic solvents like ethanol, methanol, isopropanol, N,N,dimethylformide, tetrahydrofuran, 2-methyltetrahydrofuran, acetonitrile, acetone, methylethylketone, methylenechloride, preferably tetrahydrofuran and water mixture or isopropylalcohol and water mixture, most preferably isopropylalcohol and water mixture.

The content of water in aqueous isopropylalcohol is in between 5 to 50% preferably 30% v/v.

Lapatinib ditosylate monohydrate so obtained is more than 99.9% pure by HPLC.

The crystalline Lapatinib ditosylate monohydrate so obtained has a mean particle size (D₅₀) ranging from about 5 μm to 15 μm and 90 volume % of the particles (D₉₀) ranging from 30 μm to 60 μm.

### ADVANTAGES

1) Lapatinib and its Pharmaceutically acceptable ditosylate salt obtained by this process is of high purity (99.9%).
2) The present process does not require any chromatographic purification.
3) In the present process the number of discrete synthetic steps is reduced.

The details of the invention are given in the examples below which are provided to illustrate the invention only and therefore should not be construed to limit the scope of the present invention.

### Example-1

### Preparation of N{3-chloro-4-[(3-fluorobenzyloxy]phenyl}-6-[5-({[2-methanesulphonyl)ethyl]amino}methyl)-2-furyl]-4-quinazolinamine. (or)

### Lapatinib Base (1)

(i) Preparation of 2-amino-5-iodobenzonitrile (7)

Into a one liter four necked round bottomed flask, acetic acid (200 mL), 2-aminobenzonitrile (30.0 g) were charged. To this reaction mass, iodinemonochloride (44 g) in acetic acid (200 mL) solution was added drop-wise at 25-35° C. The reaction mass was maintained at 25-35° C. for about 3 hrs. The completion of the reaction was monitored by TLC. The reaction mass was poured into ice cold water, stirred for 1 hour and filtered and dried under vacuum to get 55.0 g of brick-red coloured powder.

Purity: 97.1% by HPLC

To enhance the purity of the product the following recrystallization process was adopted.

### Purification:

Into a two liter four necked round bottomed flask, 275 mL of toluene and 55 g of crude 2-aminobenzonitrile as obtained above were charged. The mass was stirred for 30 min and clarified with activated carbon (5 g) and filtered. To the filtrate 825 mL of hexane was added and stirred for 1 hr. at 25-30° C. to crystallize out the product. The product was filtered and dried under vacuum at 30-40° C. to get 46.5 g of 2-amino-5-iodobenzonitrile as a pinkish coloured crystalline powder.

Melting-range: 85 to 87° C.

Purity: 99.89% by HPLC

(ii) Preparation of N′-(2-cyano-4-iodo-phenyl)-N,N-dimethylformamidine (8):

Into a 500 ml 3 necked round bottomed flask, N,N-dimethylforamide (100 ml), 2-amino-5-iodobenzonitrile (50 g) obtained by the process given in the above step (i) and N,N-dimethyl formamide dimethyl acetate (51.3 g) were charged. The reaction mass was maintained at 70-75° C. for about 2 Hrs. The completion of the reaction was monitored by TLC. The reaction mass was cooled to 25-35° C. and quenched into ice water and maintained for about 2 Hrs. at 0-5° C. The product was filtered and dried under vacuum at 25-35° C. to get 58 g of N′-(2-cyano-4-iodo-phenyl)-N,N-dimethylformamidine as a pale yellow coloured amorphous powder.

Melting range: 53-55° C.

HPLC purity: 99.8%

(iii) Preparation of N-[3-chloro-4-[(3-fluorobenzyloxy)phenyl]-6-iodo-quinazolin amine (3)

Into a one liter four-necked round bottomed flask, 500 mL of acetic acid, 50.0 g of N′-(2-cyano-4-iodo-phenyl)-N,N-dimethyl formamidine of the formula-(8) obtained according to the process given in the above step (ii) 51.0 g 3-chloro-4-(3′-fluorobenzyloxy)aniline were charged. The reaction mass was maintained at 115-120° C. for about 2 Hrs., the cooled to 25-35° C. and quenched into ice cold water. The pH of the reaction mass was adjusted to basic by slow addition of aqueous ammonia solution and the reaction mass was filtered and dried at 70-75° C.

Dry weight: 93.0 g

Purity: 83.64% by HPLC

The purity of the product is enhanced by adopting the following procedure.

Purification: In to a 1.0 Lt four necked round bottomed flask, 450 ml of methanol, 90 g of crude product as obtained above were charged. The reaction mass was maintained at reflux temperature for about one hour, cooled to 25-35°, filtered and dried at 70-75° to get 77 g of N-[3-chloro-4-[(3-fluorobenzyloxy)phenyl]-6-iodo-quinazolinamine (3) as a pale yellow coloured crystalline powder.

Melting range: 223-225° C.

Purity by HPLC: 99.39%

(iv) Preparation of 5-[4-[3-chloro-4-(3-fluorobenzyloxy)-anilino]-6-quinazolinyl)-furan-2-carbaldehyde (4)

Into a two liter four-necked round bottomed flask, 1000 mL of 1,2-dimethoxyethane, 50.0 g of N-(3-chloro-4-(3-fluorobenzyloxy)phenyl)-6-iodo-quinazolinamine obtained from the previous step (iii), 5-formyl-2-furyl boronic acid (21.5 g), triethylamine (30.5 g), 10% Pd on carbon (wet) (2.5 g) suspended in 500 mL of methanol were charged under stirring. The mass was maintained at 45-50° C. for about 15 hours under nitrogen atmosphere and the completion of the reaction was monitored by TLC. The catalyst was filtered and the filtrate was quenched into two liters of water and stirred well. The product was filtered and dried to get 45.0 g (96% of theory) of 5-[4-3-chloro-4-(3-fluorobenzyloxy)-anilino]-6-quinazolinyl)-furan-2-carbaldehyde as a greenish yellow amorphous powder.

Purity: 99.6% by HPLC

Melting range: 224-228° C.

(v) Preparation of N-{3-chloro-4-[(fluorobenzyloxy]phenyl}-6-[5-({[2-methane-sulphonyl)ethyl]imino}methyl)-2-furyl]-4-quinazolinamine (9)

Into a two liter four-necked round bottomed flask, 1000 mL of methanol, 40.0 g of N-(3-chloro-4-(3-fluorobenzyloxy)anilino)-6-quinazolinyl)-furan-2-carbadehyde, obtained from the previous step (iv), 20.6 g of 2-methanesulfonylethylamine HCl and 13.4 g of triethylamine were charged under stirring. The mass was maintained at reflux temperature for about 12 hours and the reaction was monitored by HPLC. The reaction mass was cooled to room temperature and filtered. The product was dried under vacuum at room temperature to get 47.0 g (96% of theory) of imine as yellow coloured crystalline solid. The product was stored under nitrogen atmosphere.

Melting point range: 74 to 76° C.

Purity: 99.0% by HPLC

Mass: 580.1 (M+1)

IR (KBr, cm⁻¹): 3339.7, 2928.0, 2362.4, 1637.3, 1608.6, 1593.4, 1572.1, 1539.1, 1497.9, 1445.9, 1425.6, 1394.3, 1371.0, 1330.5, 1294.8, 1216.9, 1198.2, 1171.6, 1123.8, 1061.4, 1026.5, 956.6, 927.8, 888.3, 868.1, 839.9, 789.7, 748.5, 679.9, 643.2, 628.7, 544.4, 517.6, 501.5.

¹H-NMR (400 MHz, DMSO-D₆): δ 3.06 (s, 3H); 3.51-3.53 (t, 2H), 3.96-3.98 (t, 2H); 5.27 (s, 2H); 7.19-7.20 (m, 2H); 7.20-7.33 (m, 4H); 7.47-7.49 (m, 1H), 7.71-7.74 (dd, 1H), 7.83-7.85 (d, 1H); 8.00-8.01 (m, 1H); 8.22-8.24 (dd, 1H); 8.33 (s, 1H); 8.58 (s, 1H); 8.86 (s, 1H) and 10.03 (s, 1H)

¹³C-NMR (400 MHz, DMSO-D₆): δ 41.84, 53.92, 54.31, 69.40, 109.25, 113.93, 114.15, 114.32, 114.61, 114.82, 115.35, 117.63, 121.08, 123.33, 124.40, 129.03, 130.62, 132.95, 139.60, 139.68, 149.52, 149.87, 151.17, 151.79, 154.80, 157.73, 161.01, 164.02, 169.05

(vi) Preparation of N{3-chloro-4-[(3-fluorobenzyloxy]phenyl}-6-[5-({[2-methanesulphonyl)ethyl]amino}methyl)-2-furyl]-4-quinazolinamine (or)

### Lapatinib Base (1)

Into a two liter four-necked round bottomed flask, 400 mL of tetrahydrofuran, 40 g of imine obtained from the previous step-(v), 400 ml of methanol were charged under stirring. The reaction mass was cooled to 0 to 5° C. and 7.0 g of sodium borohydride was added in lots and the reaction mass was maintained for about 4 hrs at 10 to 15° C. The completion of the reaction was monitored by HPLC. To this reaction mass 800 ml of water was added and the product was extracted into ethylacetate. The organic layer was separated and the solvent distilled off completely under vacuum. The solvent was distilled off completely under vacuum. The residue was cooled to 25-35° C. and 80 mL of ethylacetate was added, stirred for 2 hrs, filtered and dried under vacuum at 40-45° C. to get 30.5 g (75% on theory) of crude Lapatinib base.

Purity: 90% by HPLC

The purity of the above product was enhanced by adopting the following procedure.

### Purification:

Into a two liter four-necked round-bottomed flask, 1200 mL of methanol, 30.0 g of Lapatinib crude base obtained as above were charged under stirring. The mass was maintained at 60-65° C. for 30-45 minutes and filtered the undissolved material. The filtrate was distilled off completely under vacuum. The mass was cooled to 25-35° C. To the residue 60 mL of methanol was added, stirred for 2 hrs, filtered and dried the product under vacuum at 40-45° C. to get 28 g of pure Lapatinib base.

Purity: 99.5% by HPLC

Melting point range: 95-98° C. (Peak maximum by DSC)

The IR spectral values of pure Lapatinib base obtained are 3484, 3304, 3058, 2924, 2815, 1922, 1653, 1592, 1574, 1552, 1526, 1503, 1490, 1457, 1422, 1385, 1366, 1338, 1319, 1288, 1268, 1215, 1201, 1162, 1133, 1094, 1060, 1028, 955, 941, 892, 868, 849, 779, 747, 681, 650, 621, 552, 520, 477 cm⁻¹.

The principle 2θ values of powder XRD of pure Lapatinib base obtained are 11.22, 14.88, 16.56, 18.96, 20.97, 21.69, 22.45, 22.85, 23.21, 23.66, 25.75, 26.67, 28.12, 32.49

(vii) Preparation of N-{3-chloro-4-[(3-fluorobenzyloxy]phenyl}-6-[5-({[2-methane sulphonyl)ethyl]amino}methyl)-2-furyl]-4-quinazolinamine ditosylate salt (or) Lapatinib ditosylate (anhydrous) 1(b)

Into a two liter four-necked round bottomed flask, 1500 mL of methanol, 25 g of Lapatinib base, obtained from the previous step-(vi) were charged. The mass temperature was raised to 60-65° C. to dissolve the solid completely, and then cooled to 45-50° C. and 18 g of p-toluenesulphonic acid monohydrate dissolved in 50 mL of methanol was added. The reaction mass was maintained at reflux condition for 3 hrs, cooled to 25-35° C. and filtered. The product was dried under vacuum at 75-80° C. to get 35 g (88% of theory) of Lapatinib ditosylate salt as an yellow crystalline solid.

Melting range: 237-239° C.

Purity: 99.8% by HPLC

(viii) Preparation of N{3-chloro-4-[(3-fluorobenzyloxy]phenyl}-6-[5-({[2-methane sulphonyl)ethyl]amino}methyl)-2-furyl]-4-quinazolinamineditosylate monohydrate (or) Lapatinib ditosylate monohydrate 1(c)

Into a two liter four-necked round bottomed flask, 1000 mL of 70% isopropylalcohol in water, 25 g of Lapatinib ditosylate salt obtained from previous step-(vii) were charged. The mass temperature was raised to 75 to 80° C. and stirred for 20-30 minutes to dissolve the product completely. Then solution was clarified by carbon treatment and filtered. The filtrate was cooled to 30-35° C. under stirring. The product was filtered and dried at 70-75° C. under vacuum till water content was around 2% w/w to get 23.0 g of yellow coloured Lapatinib ditosylate monohydrate.

Purity: 99.9% by HPLC

Water content: 2.0% w/w (1.91% w/w by theory)

Particle size range: D₅₀: 5-15 μm and D₉₀: 30-60 μm

1. A process for the preparation of Lapatinib (N-{3-chloro-4-[(3-fluorobenzyloxy]phenyl}-6-[5-({[2-(methanesulfonyl)ethyl]amino}methyl]-2-furyl]-4-quinazolinamine) having the formula-(1) 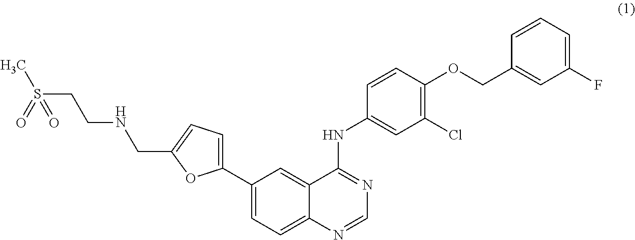
and its pharmaceutically acceptable salts
which comprises:—
(i) Reacting 2-aminobenzonitrile of formula-(6) 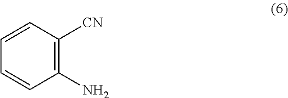
with iodinemonochloride or iodine crystals in acetic acid medium at elevated temperature to get 2-amino-5-iodobenzonitrile of the formula-(7), which is purified by recrystallization from an organic solvent or a mixture of solvents. 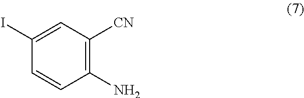
(ii) Reacting 2-amino-5-iodobenzonitrile of formula-(7) with N,N-dimethylformamide dimethyl acetal in an organic solvent and at an elevated temperature yielding the compound N′-(2-cyano-4-iodo-phenyl)-N,N-dimethyl formamidine of the formula-(8) 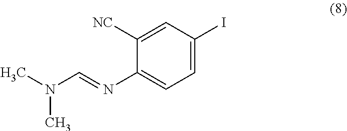
(iii) Reacting 3-chloro-4-(3-fluorobenzyloxy)aniline of formula-(8a) 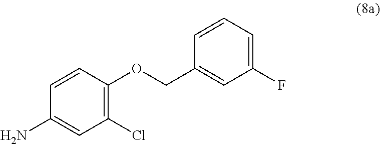
with the compound of the formula-(8) in presence of an acid catalyst and at an elevated temperature to get a compound of the formula-(3). 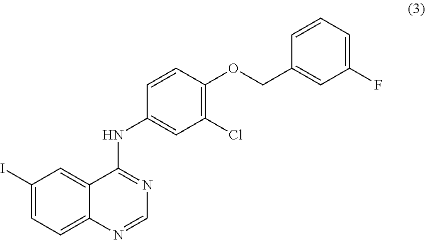
(iv) Reacting the compound of the formula-(3) with 5-formyl-2-furyl boronic acid by Palladium (0) mediated biaryl coupling (Suzuki cross coupling) in an ethereal solvent at an elevated temperature to get the desired compound of formula-(4). 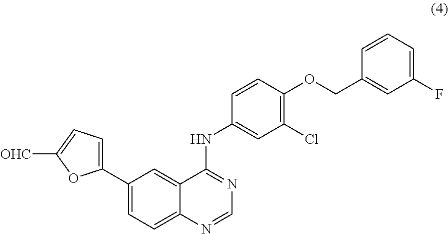
(v) Reacting the compound of the formula-(4), with 2-methane sulfonylethylamine or its salt in a suitable solvent, at an elevated temperature gives the imine compound of the formula-(9). 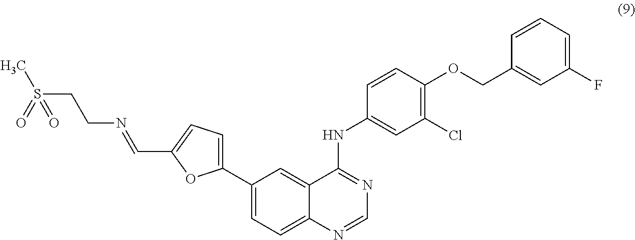
(vi) Reacting the compound of the formula-(9) with a suitable reducing agent in a suitable solvent and the resultant amine formed is extracted with a suitable solvent and subsequent evaporation of the solvent affords Lapatinib base of the formula-(1) 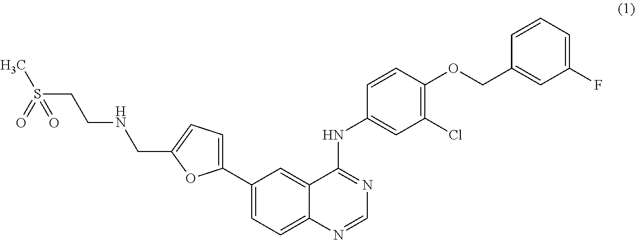
(vii) Crystallizing the crude Lapatinib base of formula-(1) from a suitable solvent to get pure Lapatinib base.
(viii) Reacting pure Lapatinib base of formula-(1) by dissolving or suspending in an organic solvent with p-toluenesulfonic acid monohydrate to get Lapatinib ditosylate (anhydrous) of formula-1(b) 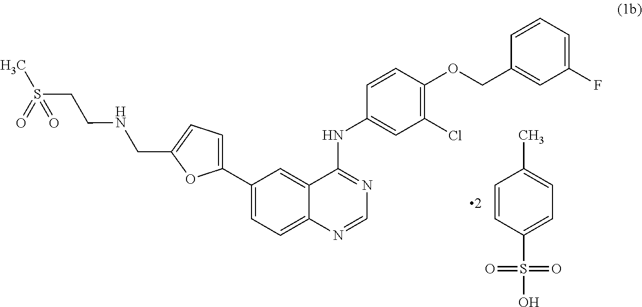
(ix) Recrystallization of Lapatinib ditosylate (anhydrous) in aqueous alcohol affords pharmaceutically acceptable grade Lapatinib ditosylate monohydrate of formula-1(c).

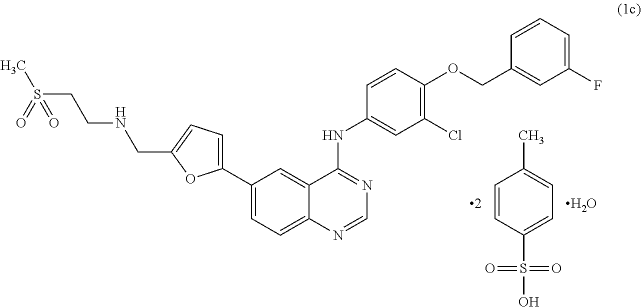

2. A process as claimed in claim 1, where in step-(i) during iodination of 2-aminobenzonitrile, the reaction temperature is maintained at 0 to 100° C., preferably between 10 to 50° C., most preferably between 25 to 35° C.

3. A process as claimed in claim 1, where in step-(i) during iodination of 2-aminobenzonitrile, the organic solvent used for purification by recrystallization, is a mixture of toluene and hexane.

4. A process as claimed in claim 1, where in step-(ii), to obtain compound of formula-(8), the reagent used in the reaction of 2-amino-5-iodobenzonitrile of formula-(7), is N,N-dimethylformamide dimethylacetal.

5. A process as claimed in claim 1, where in step-(ii), the solvent used for the reaction is selected from toluene, xylene, cumene, chlorobenzene, acetonitrile, propionitrile, butyronitrile, ethylacetate, tetrahydrofuran, 2-methyltetrahydrofuran, 1,4-dioxane, N,N-dimethylformamide, N,N-dimethyacetamide, N-methyl-pyrrolidin-2-one, dimethylsulfoxide, water, methanol, ethanol, 2-propanol, butanol or pentanol or mixtures thereof. Conveniently N,N-dimethylformamide, N,N-dimethyacetamide, N-methyl-pyrrolidin-2-one, dimethylsulfoxide preferably N,N-dimethylformamide.

6. A process as claimed in claim 1, where in step-(ii), the reaction temperature is in between 30 to 150° C., preferably between 70 to 80° C.

7. A process as claimed in claim 1, where in step-(ii), the product is isolated by quenching the reaction mass into water.

8. A process as claimed in claim 1, where in step-(ii), the product isolation temperature is in between 0 to 40° C., preferably 0 to 5° C.

9. A process as claimed in claim 1, where in step-(iii), the compound of formula-(8) is coupled with compound of formula-(8a) in the presence of acid catalyst, which is selected from trifluoroacetic acid, formic acid or acetic acid, preferably acetic acid.

10. A process as claimed in claim 1, where in step-(iii), the reaction temperature is in between 30-140° C., preferably 115-120° C.

11. A process as claimed in claim 1, where in step-(iii), the product is obtained by quenching the reaction mass into water and adjusted the pH to basic by adding aqueous ammonia or dilute caustic lye solution.

12. A process as claimed in claim 1, where in step-(iii), the product isolation temperature is in between 0-40° C. preferably O-5° C.

13. A process as claimed in claim 1, where in step-(iv), in Suzuki coupling reaction, the ethereal solvent used is selected from diethylether, tetrahydrofuran, 1,4-dioxane, 1,2-diethoxyethane and 1,2-dimethoxyethane, preferably 1,2-dimethoxyethane (DME).

14. A process as claimed in claim 1, where in step-(iv), the catalyst used is selected from a list that includes palladium (II) acetate, palladium (II) chloride, palladium on carbon, preferably palladium on carbon.

15. A process as claimed in claim 1, where in step-(iv), the reaction temperature is in between 25 to 120° C. preferably between 25 to 75° C. and most preferably between 45-50° C.

16. A process as claimed in claim 1, where in step-(v), the aldehyde compound of the formula-(4) is reacted with 2-methanesulphonyl ethylamine or its salts with acids like HCl, HBr or H₂SO₄, preferably HCl salt.

17. A process as claimed in claim 1, where in step-(v), the solvent used for the reaction includes dichloroethane, dichloromethane, tetrahydrofuran, 2-methyl tetrahydrofuran, N,N-dimethylformamide, 1,2-dimethoxyethane and alcohols like ethanol, methanol, 2-propanol or a mixture thereof. The preferred solvents are tetrahydrofuran and methanol, most preferably methanol.

18. A process as claimed in claim 1, where in step-(v), the reaction temperature is in between 0 to 125° C. preferably between 25 to 100° C. and most preferably the reflux temperature of methanol.

19. A process as claimed in claim 1, where in step-(vi), for the reduction of imine of formula-(9) to amine, the reducing agent used is selected from sodium triacetoxyborohydride, sodium borohydride etc, preferably sodium borohydride.

20. A process as claimed in claim 1, where in step-(vi), the solvent used in the reaction can be selected from tetrahydrofuran, acetonitrile, acetone, dimethylformamide, dimethylacetamide, 1,2-diethoxyethane, 1,2-dimethoxyethane or a mixture thereof, preferably a mixture of tetrahydrofuran and methanol.

21. A process as claimed in claim 1, where in step-(vi), the reaction temperature is in between 0 to 100° C. preferably 0 to 40° C. most preferably 0 to 15° C.

22. A process as claimed in claim 1, where in step-(vi), the solvent for extraction of the product of formula-(1) is selected form ethylacetate, methylacetate, isopropylacetate, tertiarybutylmethylether, dichloroethane, dichloromethane, chloroform etc. preferably ethylacetate.

23. A process as claimed in claim 1, where in step-(vii), the solvent used for purification of Lapatinib crude base by crystallization is selected from ethylacetate, methylacetate, isopropylacetate, acetonitrile, methanol, ethanol, isopropanol, acetone, methylethylketone, methylenechloride, toluene, chloroform, 1,4-dioxane, dimethyl formamide, tetrahydrofuran, 2-methyltetrahydrofuran, dimethylacetamide, 1,2-dimethoxyethane, tertiarybutylmethylether, water or a mixture thereof, preferably ethylacetate, isopropanol and methanol.

24. A process as claimed in claim 1, where in step-(vii), the isolation temperature of crystallized Lapatinib base is in between 0 to 35° C. preferably 25-35° C.

25. A process as claimed in claim 1, where in step-(vii), the purity of Lapatinib base obtained according to process of the present invention is more than 99.5% by HPLC.

26. A process as claimed in claim 1, where in step-(vii), The melting point range of the pure Lapatinib base obtained is 95-98° C. (Peak maximum by DSC).

27. A process as claimed in claim 1, where in step-(vii), the 2θ values of powder XRD of pure Lapatinib base obtained are 11.22, 14.88, 16.56, 18.96, 20.97, 21.69, 22.45, 22.85, 23.21, 23.66, 25.75, 26.67, 28.12, 32.49.

28. A process as claimed in claim 1, where in step-(vii), the IR spectral values of pure Lapatinib base obtained are 3484, 3304, 3058, 2924, 2815, 1922, 1653, 1592, 1574, 1552, 1526, 1503, 1490, 1457, 1422, 1385, 1366, 1338, 1319, 1288, 1268, 1215, 1201, 1162, 1133, 1094, 1060, 1028, 955, 941, 892, 868, 849, 779, 747, 681, 650, 621, 552, 520, 477 cm⁻¹.

29. A process as claimed in claim 1, where in step-(viii), the purified Lapatinib base so obtained can be converted into ditosylate salt (anhydrous) by suspending or dissolving the Lapatinib base in an organic solvent or a mixture of organic solvents and then treating with p-toluene sulfonic acid monohydrate.

30. A process as claimed in claim 1, where in step-(viii), the organic solvent used for dissolving or suspending pure Lapatinib base is selected from toluene, chloroform, isopropanol, ethanol, methanol, acetone, methyethylketone, acetonitrile, methylacetate, ethylacetate, isopropylacetate, dimethylformamide, dimethylether, diethylether, tertiarybutylmethylether, tetrahydrofuran, 2-methyltetrahydrofuran, dimethylacetamide, 1,2-diethoxyethane, 1,2-dimethoxyethane or a mixture thereof, preferably tetrahydrofuran, methanol or most preferably methanol.

31. A process as claimed in claim 1, where in step-(viii), the reaction temperature during ditosylate salt formation is in between 0 to 80° C., preferably the refluxing temperature of the solvent used.

32. A process as claimed in claim 1, where in step-(viii), the isolation temperature of ditosylate salt formed is in between 0 to 35° C. preferably 25-35° C.

33. A process as claimed in claim 1, where in step-(ix), Lapatinib ditosylate monohydrate is obtained by suspending or dissolving Lapatinib ditosylate (anhydrous) in a mixture of water and organic solvents like ethanol, methanol, isopropanol, N,N,dimethylformide, tetrahydrofuran, 2-methyltetrahydrofuran, acetonitrile, acetone, methylethylketone, methylenechloride, preferably tetrahydrofuran and water mixture or isopropylalcohol and water mixture, most preferably isopropylalcohol and water mixture.

34. A process as claimed in claim 1, where in step-(ix), the content of water in aqueous isopropyl alcohol is in between 5 to 50% preferably 30% v/v.

35. A process as claimed in claim 1, where in step (ix), the Lapatinib ditosylate monodihydrate obtained is of purity more than 99.9% by HPLC.

36. A process as claimed in claim 1, where in step-(ix), the crystalline Lapatinib ditosylate monodihydrate so obtained has a particle size (D₅₀) ranging from about 5 μm to 15 μm and 90 volume % of the particles (D₉₀) ranging from 30 vim to 60 μm.

37. (canceled)
